(19) United States
(12) Patent Application Publication
(10) Pub. No.: US 2011/0060292 A1
(43) Pub. Date: Mar. 10, 2011

(54) PEN NEEDLE STORAGE DEVICE WITH INTEGRAL REMOVAL AND/OR INSTALLATION SYSTEM

(75) Inventor: Steven SCHRAGA, Surfside, FL (US)

(73) Assignee: STAT MEDICAL DEVICES, INC., North Miami, FL (US)

(21) Appl. No.: 12/855,358

(22) Filed: Aug. 12, 2010

Related U.S. Application Data
(60) Provisional application No. 61/234,107, filed on Aug. 14, 2009.

(51) Int. Cl. A61M 5/34; A61B 19/02

(52) U.S. Cl. 604/240; 206/366

(57) ABSTRACT

A device for at least one of storing, installing and removing pen needles. The device includes a body configured to store at least one pen needle. A first end of the body is configured to install a pen needle on an injection device. A second end of the body is configured to remove a pen needle from the injection device. The invention also provides for a method of installing and/or removing a pen needle from an injection device using the device. This Abstract is not intended to define the invention disclosed in the specification, nor intended to limit the scope of the invention in any way.

### CROSS-REFERENCE TO RELATED APPLICATIONS

The instant application is a U.S. non-provisional Application based on U.S. provisional application No. 61/234,107, filed Aug. 14, 2009, the disclosure of which is hereby expressly incorporated by reference hereto in its entirety.

### BACKGROUND OF THE INVENTION

1. Field of the Invention

This invention relates generally to the field of skin puncturing devices. Such devices include a pen injection devices, e.g., pre-loaded syringes, such are utilized for injection of medicament into the body tissues of human and animal patients. More specifically, this invention relates to a removable pen needle storage device which makes in easier and safer to removed the pen needle from the pen injection device and/or to install the pen needle on the pen injection device. The pen needle storage device can preferably include a storage area which safely stores multiple unused pen needles as well as used pen needles.

This invention also relates to a method of using the pen needle storage device to install and remove pen needles from pen injection devices wherein the storage device is configured to be used with conventional pen needles and pen injection devices.

This invention also relates to a method of installing and/or removing pen needles from pen injection devices more safely, i.e., preventing the user from being pricked when handling and/or attempting to remove the pen needle from the pen injection device.

2. Discussion of Background Information 100051 Pen needles are known from documents such as U.S. 2008/0154192, the disclosure of which is hereby expressly incorporated by reference in its entirety. However, devices which can safely store unused pen needles, install the unused pen needles, and/or remove and store the used pen needles are believed to be unknown.

It is therefore desirable to provide a pen needle storage, installation, and removal system which makes it safer to install, store, and remove pen needles.

### SUMMARY OF THE INVENTION

According to one non-limiting embodiment of the invention, there is provided a device for at least one of storing, installing and removing pen needles, wherein the device comprises a body configured to store at least one pen needle, a first end of the body being configured to install a pen needle on an injection device, and a second end of the body being configured to remove a pen needle from the injection device.

The at least one pen needle may comprise at least two pen needles. The at least one pen needle may comprise at least three pen needles. The at least one pen needle may comprise between four and eight pen needles. The body may be generally cylindrical. The first end of the body may comprise at least one mechanism for releasably retaining the at least one pen needle which releasably retains the at least one pen needle until it is fully installed on the injection device. The first end of the body may comprise plural mechanisms for releasably retaining the at least one pen needle which releasably retains the at least one pen needle until it is fully installed on the injection device. The first end of the body may comprise at least one mechanism for releasably retaining the at least one pen needle in an installation position. The first end of the body may comprise plural mechanisms for releasably retaining the at least one pen needle in an installation position. The second end of the body may comprise at least one mechanism for releasably retaining the at least one pen needle during removal of the pen needle from the injection device. The first end of the body may comprise plural mechanisms for releasably retaining the at least one pen needle during removal of the pen needle from the injection device. The second end of the body may comprise at least one mechanism for releasably retaining the at least one pen needle in a removal position. The first end of the body may comprise plural mechanisms for releasably retaining the at least one pen needle in a removal position.

The first end of the body may be configured to retain the at least one pen needle while a needle of the at least one pen needle is oriented into the body. The first end of the body may be configured to retain the at least one pen needle while a needle of the at least one pen needle is oriented towards the second end. The second end of the body may be configured to retain the at least one pen needle while a needle of the at least one pen needle is oriented out of the body. The second end of the body may be configured to retain the at least one pen needle while a needle of the at least one pen needle is oriented opposite the first end. The first end of the body may be configured to retain a new or unused pen needle. The second end of the body may be configured to retain a used pen needle.

The body may be configured to retain a new or unused pen needle in the first end while the at least one pen needle is arranged between the first and second ends. The body may be configured to retain a used pen needle in the second end while the at least one pen needle is arranged between the first and second ends. While the at least one pen needle is arranged between the first and second ends, the body may be configured to retain a new or unused pen needle in the first end and retain a used pen needle in the second end. Wherein, while the at least one pen needle is arranged between the first and second ends, the body may be configured to releasably retain a new or unused pen needle in the first end and non-removably retain a used pen needle in the second end. 100111 The invention also provides for a method of installing a pen needle on an injection device using the device described above, wherein the method comprises arranging the first end adjacent a pen needle receiving end of the injection device and moving the device towards the injection device until the pen needle is installed thereon.

The invention also provides for a method of method of removing a pen needle from an injection device using the device described above, wherein the method comprises arranging the second end adjacent a pen needle receiving end of the injection device and moving the device towards the injection device until the pen needle is installed in the second end.

The invention also provides for a method of installing a pen needle on an injection device using the device described above, wherein the method comprises arranging the first end adjacent a pen needle receiving end of the injection device, moving the device towards the injection device until the pen needle is installed thereon, and moving the device away from the injection device with one fewer pen needle being arranged in the body than prior to the arranging.

The invention also provides for a method of removing a pen needle from an injection device using the device described above, wherein the method comprises arranging the second end adjacent a pen needle receiving end of the injection device, moving the device towards the injection device until the pen needle is installed in the second end, and moving the device away from the injection device with one additional pen needle being arranged in the body than prior to the arranging.

The invention also provides for a device for storing and at least one of installing and removing pen needles, wherein the device comprises an elongate body configured to store plural pen needles, a first end of the body being configured to install a pen needle on an injection device, and a second end of the body being configured to remove a pen needle from the injection device.

The invention also provides for a device for storing, installing and removing pen needles, wherein the device comprises a body configured to store plural pen needles, a first end of the body being configured to install a pen needle on an injection device, a second end of the body being configured to remove a pen needle from the injection device, and a movable device arranged in the body adapted to separate at least one unused pen needle from a used pen needle.

The invention also provides for a device for at least one of storing, installing and removing skin puncturing devices, wherein the device comprises a body configured to store at least one skin puncturing device, a first end of the body being configured to install a skin puncturing device, and a second end of the body being configured to remove a skin puncturing device.

The at least one skin puncturing device may comprise at least one pen needle. The device may be disposable after being used at least one time. The device may be rendered un-usable after being used a predetermined number of times. The device may be re-fillable after being used at least one time.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a side cross-section view of a first non-limiting embodiment of a pen needle storage, installation and removal device according to the invention. The pen needles are not shown in cross-section. However, a safety cap of each pen needle is shown in cross-section;

FIG. 2 shows the device of FIG. 1 being used to remove a used pen needle from an injection device;

FIG. 3 shows the device of FIG. 1 after it is used to remove a used pen needle from an injection device;

FIG. 4 shows the device of FIG. 3 being used to install an unused or new pen needle on an injection device;

FIG. 5 shows the device of FIG. 3 while it is used to remove a second used pen needle from an injection device;

FIG. 6 shows the device of FIG. 5 while it is used to remove a third used pen needle from an injection device;

FIG. 7 shows the device of FIG. 1 being used to install an unused or new pen needle on an injection device;

FIG. 8 shows the device of FIG. 7 after being used to install the first unused or new pen needle on an injection device;

FIG. 9 shows the device of FIG. 8 while it is used to remove a first used pen needle from an injection device;

FIGS. 10 and 11 show the device of FIG. 9 while it is used to install a second unused or new pen needle on an injection device;

FIG. 12 shows the device of FIG. 1 after it is used to install all of its unused pen needles and used to remove and store a maximum number of used pen needles;

FIG. 13 shows an enlarged partial side cross-section view of a second non-limiting embodiment of a pen needle storage, installation and removal device according to the invention. The pen needles are not shown in cross-section. However, a safety cap of each pen needle is shown in cross-section;

FIG. 14 shows one of the pen needles used in the embodiment of FIG. 13;

FIGS. 15-17 show one of the pen needle safety caps used in the embodiment of FIG. 13;

FIG. 18 shows the body assembly used in the embodiment of FIG. 1;

FIG. 19 shows the body used in the body assembly of FIG. 18;

FIG. 20 shows the body of FIG. 19 rotated 90 degrees and not in cross-section;

FIGS. 21-23 show a separator device used in the body assembly of FIG. 18;

FIG. 24 shows a spring used in the body assembly of FIG. 18;

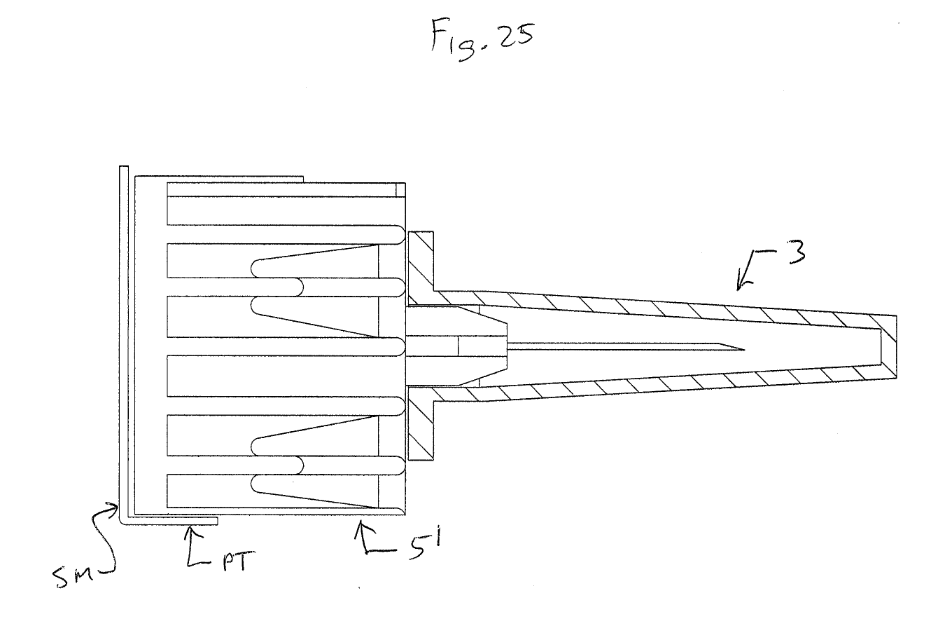

FIGS. 25-27 show side and end views of an embodiment of the pen needles that can be used in the embodiment of FIG. 1;

FIG. 28 shows a side cross-section view of another non-limiting embodiment of a pen needle storage, installation and removal device according to the invention. The pen needles are not shown in cross-section. However, a safety cap of each pen needle is shown in cross-section;

FIGS. 29 and 30 show partial side cross-section views of a removal end of another non-limiting embodiment of a pen needle storage, installation and removal device according to the invention. In FIG. 29, a hand-actuated slide mechanism is in an initial position. In FIG. 30, the hand-actuated slide mechanism is in a position which causes inwardly deflected members to move to a position that can engaged with a used pen needle (not shown);

FIGS. 31 and 32 shows a partial side cross-section view of a removal end of another non-limiting embodiment of a pen needle storage, installation and removal device according to the invention. In FIG. 32, a hand-actuated slide mechanism is in an initial forward position. In FIG. 31, the hand-actuated slide mechanism is in a retracted position which allows inwardly deflected members to move to a position that can engaged with a used pen needle (not shown); and

FIGS. 33 and 34 show side views of another non-limiting embodiment of a pen needle storage, installation and removal device according to the invention. In FIG. 33, the pusher member is located in a position indicating that five new or unused pen needles remain in the device. In FIG. 34, the pusher member is located in a position indicating that three new or unused pen needles remain in the device.

### DETAILED DESCRIPTION OF THE INVENTION

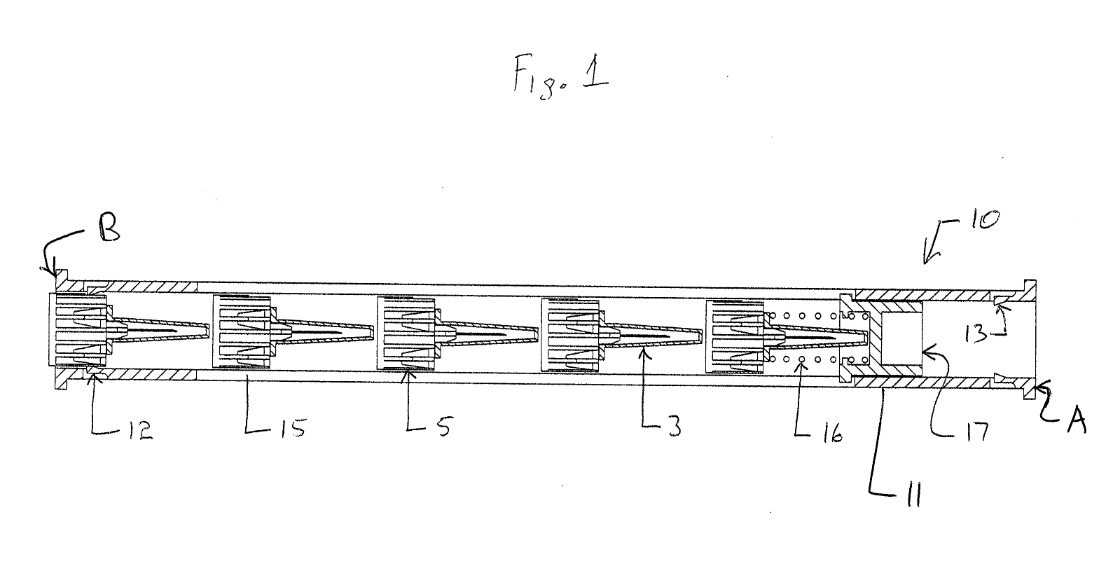

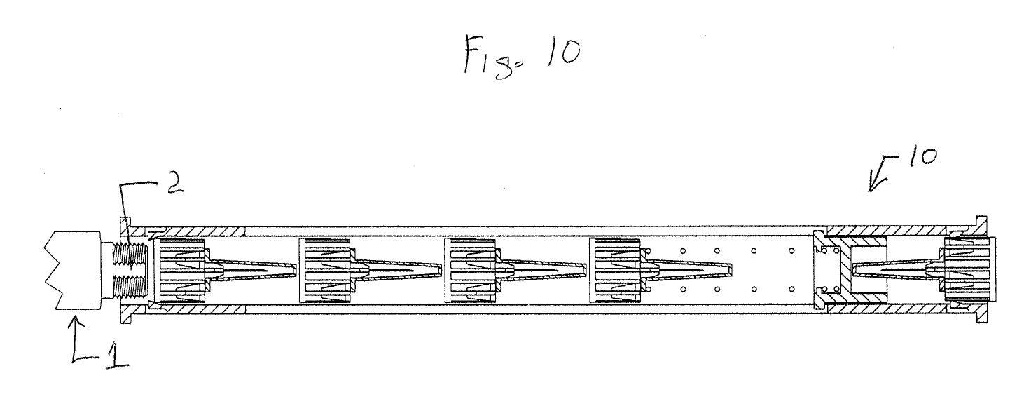

Referring now to the drawings and first to FIGS. 1-12 which shows a first embodiment of a pen needle storage, installation and removal device according to the invention. The device 10 is sized and configured to receive therein a number of new or unused pen needles 5, as well as number of used pen needles 5 when the device 10 reaches its useful life. Each pen needle 5 includes a removable needle cover 3. By way of non-limiting example, the device 10 is sized and configured to receive therein five pen needles 5. The pen needle 5 may be of any type such as those conventionally known in the art. The proximal end of the pen needle 5 includes a needle while the distal end includes an opening which is sized to allow the pen needle 5 to be mounted to the threaded proximal end 2 of the pen needle injection device 1 (see FIG. 10).

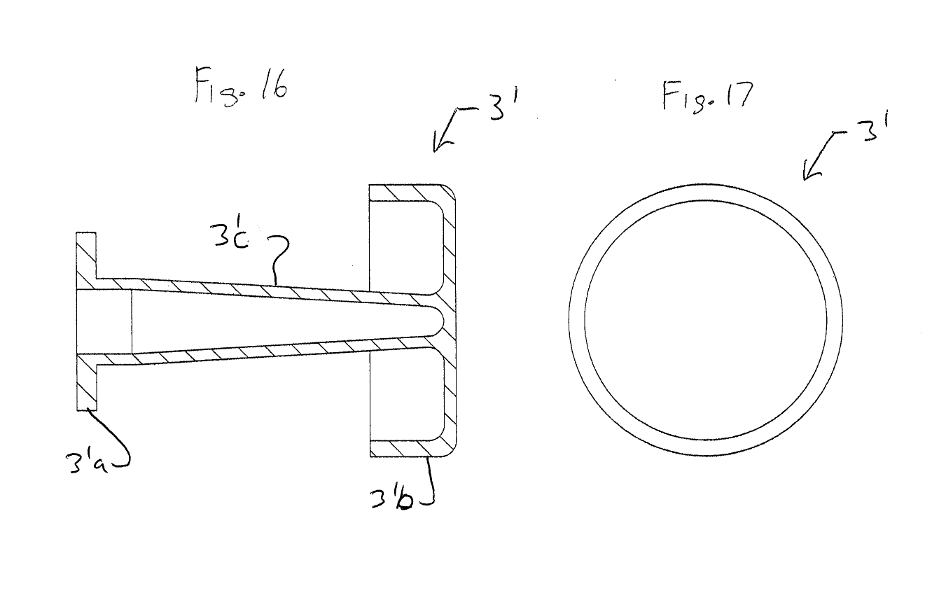

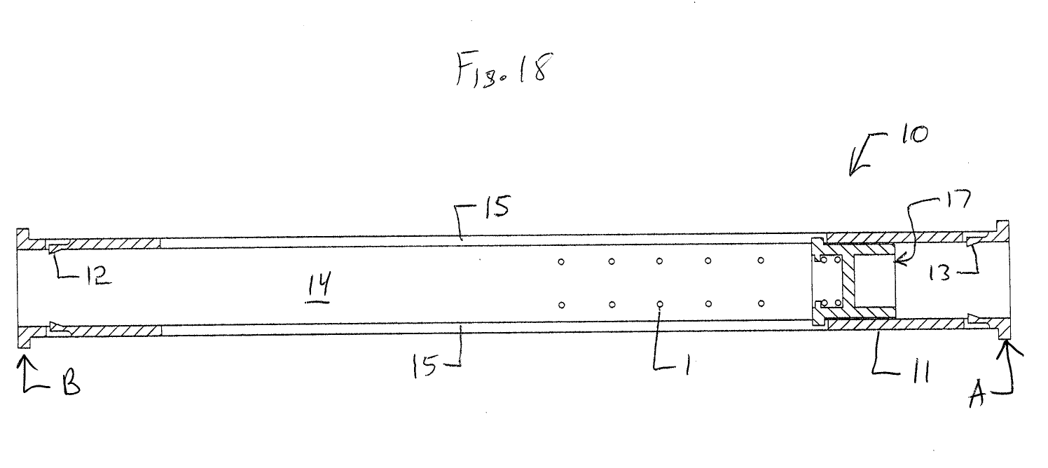

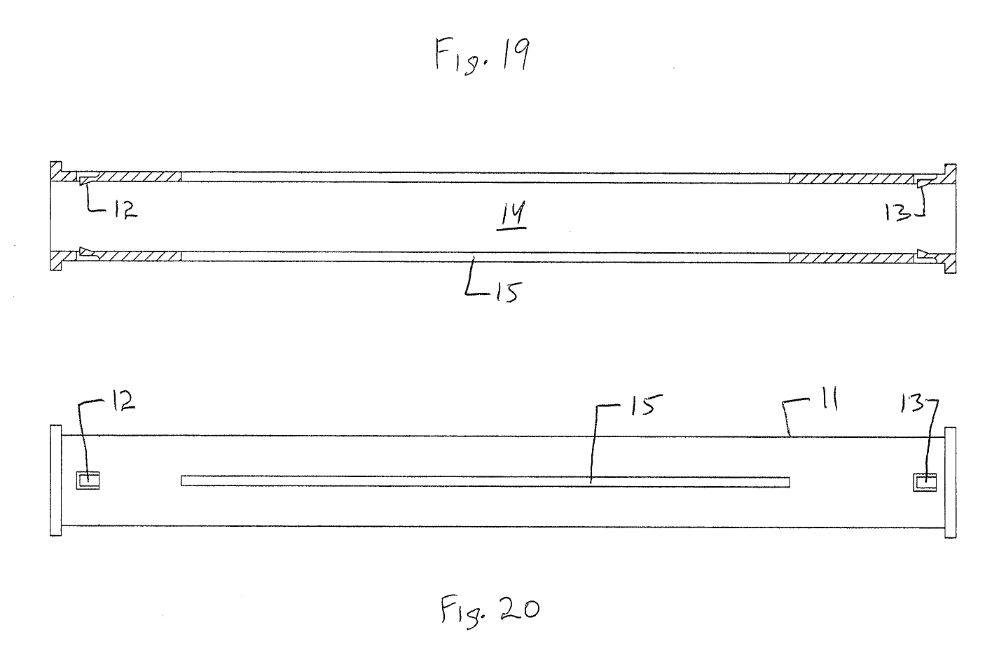

The device 10 comprises a main elongated generally cylindrical body 11 having a first end A and a second end B, as well as a number of features shown in FIGS. 18-20 which are described below.

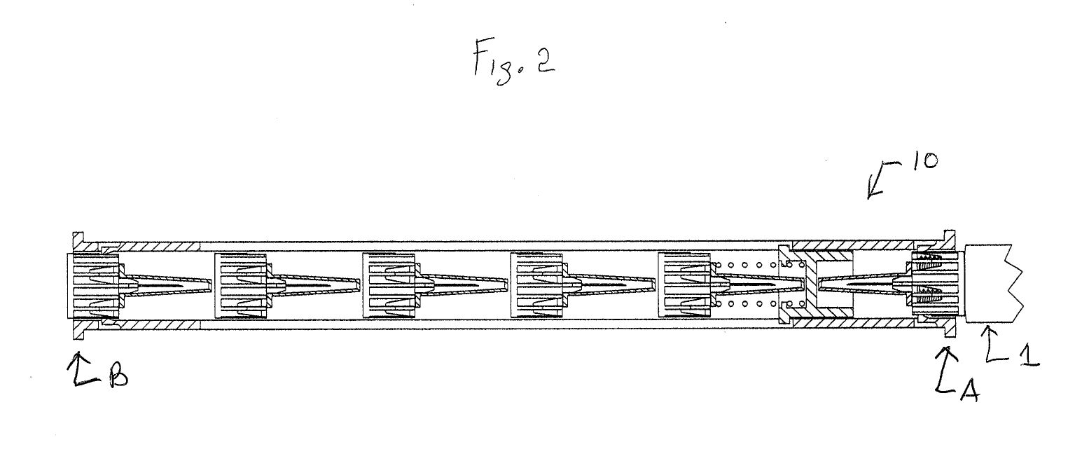

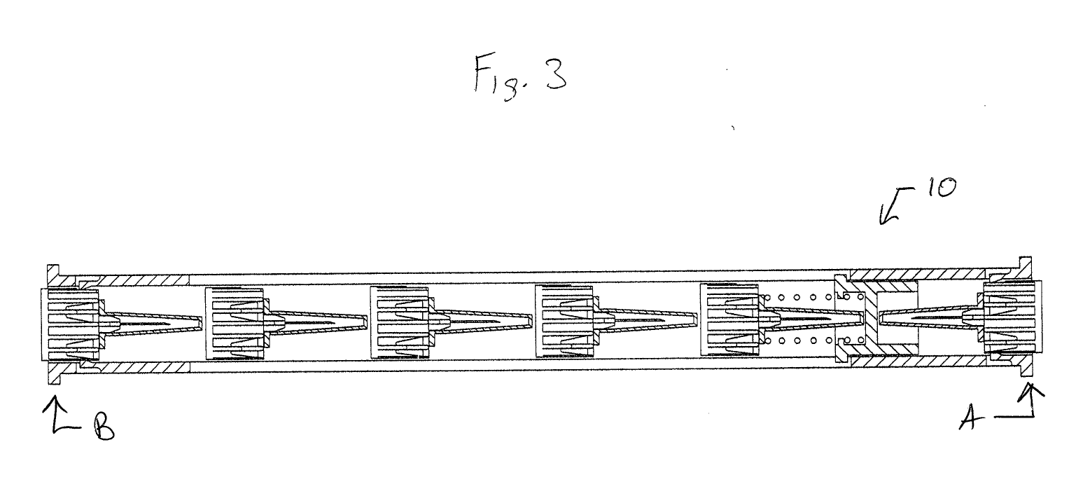

The first end A is sized and configured to be utilized to remove a used pen needle 5 from an injection device 1. That is, the end A has an opening that can receive therein a used pen needle 5 (see FIGS. 2-3). End A also includes plural engaging projections 13 which frictionally engage with the body of the pen needle 5 such that rotation of the body 11 results in a corresponding rotation of the pen needle 5 received in the end A. When the user places the installed pen needle 5 (i.e., installed on the device 1) as shown in FIG. 2 and then rotates either the device 1 or the device 10 along a direction that causes an unthreading of the pen needle 5 from the device 1, the device 10 can be used to safely remove the pen needle 5 from the device 1 (see FIG. 3). The removed pen needle 5 is then remains safely stored in the body 11.

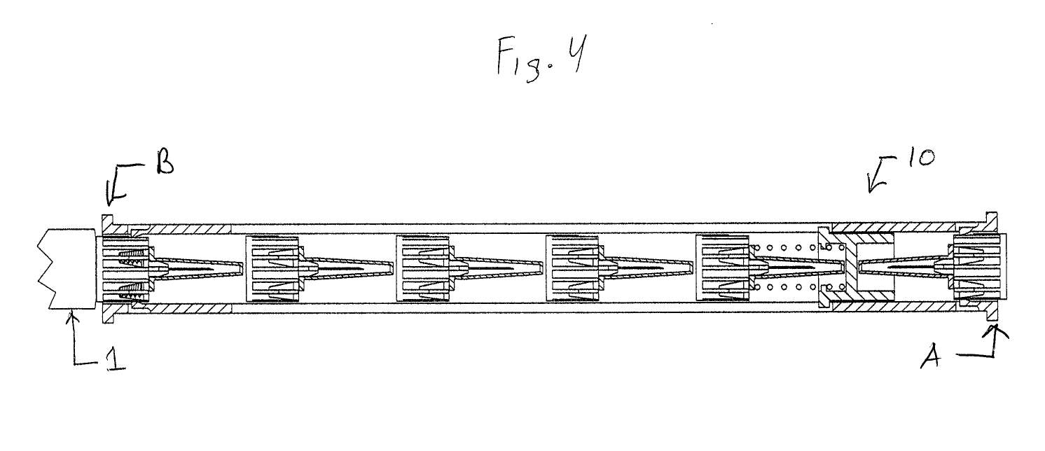

The second end B is sized and configured to be utilized to install a new pen needle 5 onto an injection device 1. That is, the end B has an opening that has arranged therein a new pen needle 5 (see FIG. 1). End B also includes plural engaging projections 12 which frictionally engage with the body of the pen needle 5 such that rotation of the body 11 results in a corresponding rotation of the pen needle 5 received in the end B. When the user places end B against the threaded section 2 of the injection device 1 and then rotates either the device 1 or the device 10 along a direction that causes a threading-on of the pen needle 5 onto the device 1, the device 10 can be used to safely install the pen needle 5 on the device 1 (see FIG. 4). The installed pen needle 5 shown in FIG. 4 can then be removed from the body 11 and used for injection.

The body 11 also comprises a main elongated generally cylindrical space 14 which receive therein the pen needles 5. One or more elongated slots 15 are formed in the side of the body 11. The slot(s) 15 function to linearly and non-rotatably guide the movement of a pusher/separator member 17. A spring 16 is also disposed in the body 11 and has one end coupled to the member 17 and another end which applies a biasing force against an adjacent pen needle 5 (see FIG. 1).

The operation of the device 10 will now be described with reference to FIGS. 1-10. The device 10 of FIG. 1 is characteristic of an initial and/or an initial prior-use configuration, i.e., having a state which can be packaged. In this position, the body 11 has stored therein plural new pen needles 5 with the spring 16 exerting a biasing force against an adjacent pen needle 5 which, in turn, places all of the new pen needles 5 inside the body 11 in slight axial compression. The area of end A between the edge of the body 11 and the proximal end of the member 17 remains essentially empty and is sized to accommodate a used pen needle 5.

The device 10 of FIGS. 2 and 3 is characteristic of one type of use configuration, i.e., having a state in which it is being used to remove a first used pen needle 5. In this position, the end A receives therein a used and installed pen needle 5. When the user places the installed pen needle 5 (i.e., installed on the device 1) as shown in FIG. 2 and then rotates either the device 1 or the device 10 along a direction that causes an unthreading of the pen needle 5 from the device 1, the device 10 can be used to safely remove the pen needle 5 from the device 1 (see FIG. 3). The removed pen needle 5 is then remains safely stored in end A of the body 11.

It should be noted that prior to the user placing the used pen needle 5 into end A of the device 10 in order to remove the same as shown in FIG. 2, the user will typically have already used the device 1 with the pen needle 5 installed thereon for injection. After injection and/or use of the pen needle 5, the user will reinstall the needle cap 3 (which is typically removed just prior to injection). Then, the used pen needle 5 with the cap 3 installed thereon can be positioned in end A as shown in FIG. 2.

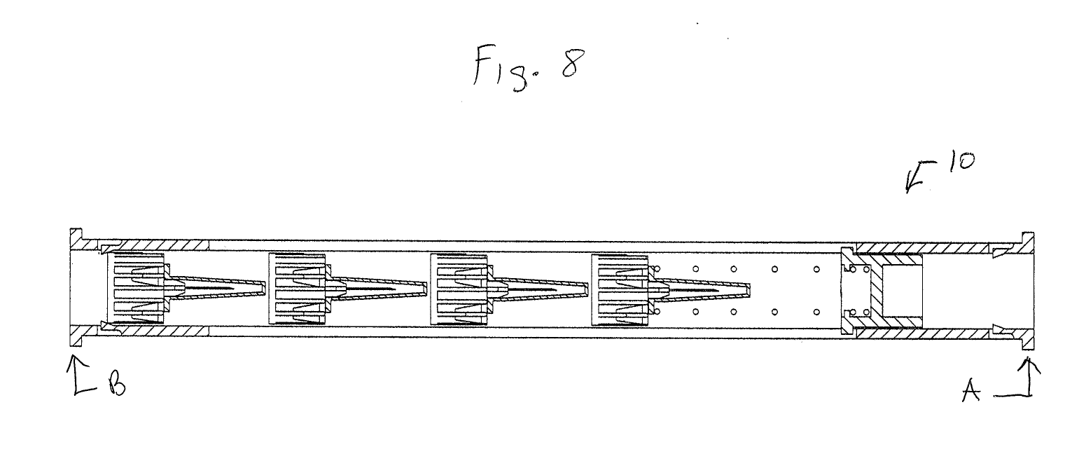

The device 10 of FIG. 4 is characteristic of another type of use configuration, i.e., having a state in which it is being used to install a first new pen needle 5. In this position, the end B receives therein a threaded section 2 of a device 1. When the user places the section 2 against the first new pen needle 5 and rotates either the device 1 or the device 11 along a direction that causes a threading-on of the pen needle 5 (until it reaches the installed position of FIG. 4), the user can then remove the device 1 from the device 10 and use the device 1 for injection. Once the first new pen needle 5 is removed from the device 10, the spring 16 will cause the second new pen needle 5 to move to a position in contact with the projections 12 (see e.g., FIG. 8).

It should be noted that prior to the user installing the first new pen needle 5 arranged in end B of the device 10 as shown in FIG. 2, the cap 3 of the first new pen needle 5 will extend partially into the body of the second new pen needle 5 and being in contact with the needle arranged therein. The small space shown in FIG. 4 between the proximal end of the cap 3 and the body of the second new pen needle 5 would not exist because of the biasing force applied by the spring 16 and is illustrated to show the free end of the needle caps 3.

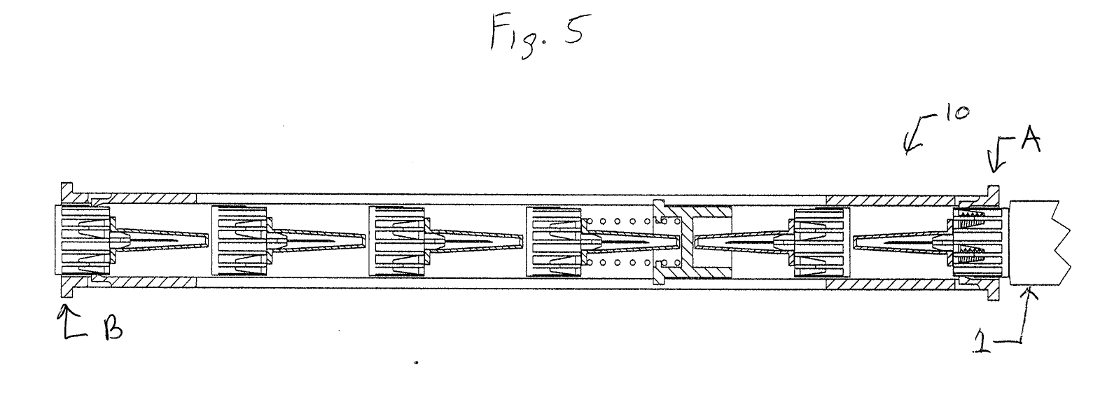

The device 10 of FIG. 5 is characteristic of another type of use configuration, i.e., having a state in which it is being used to remove a second used pen needle 5. In this position, the end A receives therein another or second used and installed pen needle 5. When the user places the installed pen needle 5 (i.e., installed on the device 1) in end A as shown in FIG. 5 and then rotates either the device 1 or the device 10 along a direction that causes an unthreading of the pen needle 5 from the device 1, the device 10 can be used to safely remove the pen needle 5 from the device 1. The removed pen needle 5 is then remains safely stored in end A of the body 11.

The user can then install a new or unused pen needle 5 on the device 1 by positioning the threaded section 2 of the device 1 against end B and use the same for injection. This leaves the device 10 with three new or unused pen needles 5 and two used pen needles 5.

It should be noted that while FIG. 5 shows the free end of the cap 3 of the second used pen needle 5 slightly spaced from the previously inserted first used pen needle 5, it reality the free end of the cap 3 will extend partially into the first used pen needle 5 and contact an end of the needle, i.e., the inside portion of the double-ended needle, within the body of the first used pen needle 5. Once this contact occurs, any movement of the second used pen needle 5 into end A causes movement of the first used pen needle 5 towards end B, which, in turn, pushed member 17 toward end B.

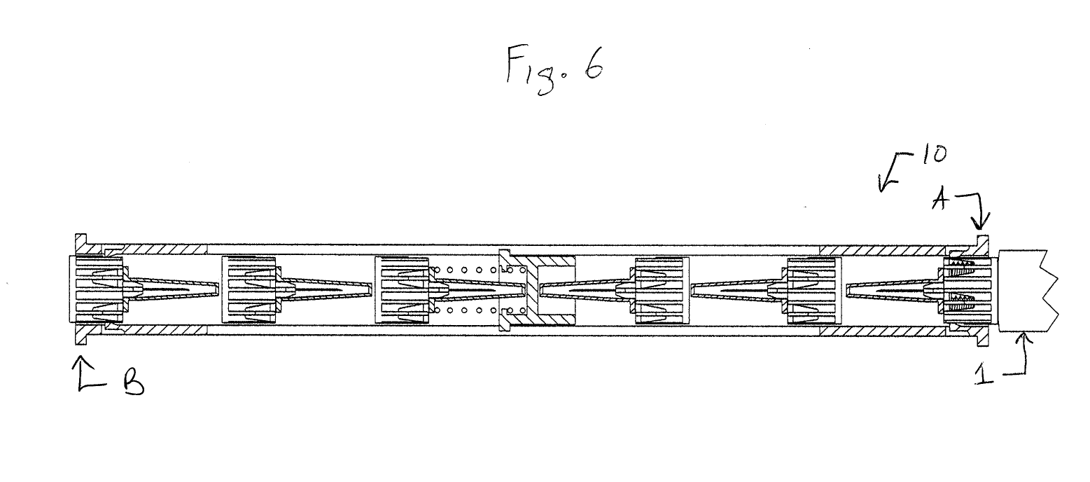

The device 10 of FIG. 6 is characteristic of still another type of use configuration, i.e., having a state in which it is being used to remove a third used pen needle 5. In this position, the end A receives therein another or third used and installed pen needle 5. When the user places the installed pen needle 5 (i.e., installed on the device 1) in end A as shown in FIG. 6 and then rotates either the device 1 or the device 10 along a direction that causes an unthreading of the pen needle 5 from the device 1, the device 10 can be used to safely remove the pen needle 5 from the device 1. The removed pen needle 5 is then remains safely stored in end A of the body 11.

The user can then install a new or unused pen needle 5 on the device 1 by positioning the threaded section 2 of the device 1 against end B and use the same for injection. This leaves the device 10 with two new or unused pen needles 5 and three used pen needles 5.

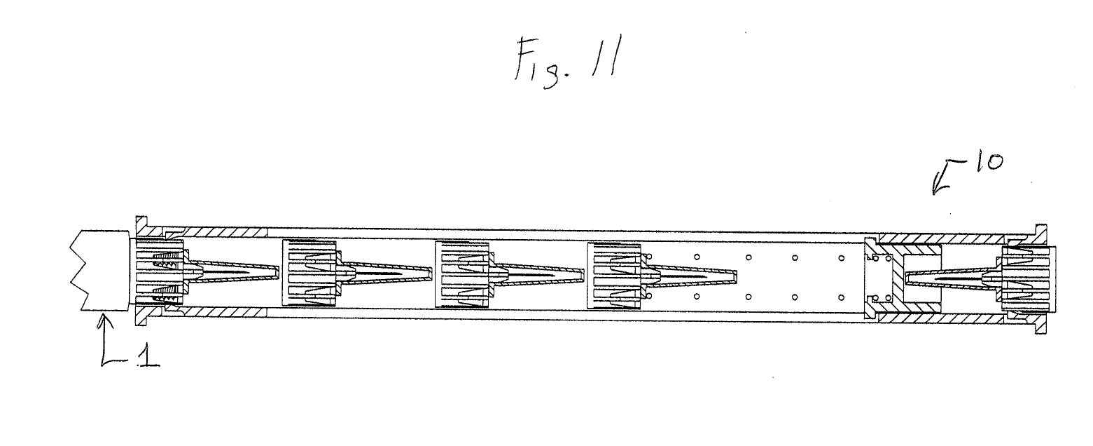

The above-noted steps are repeated until all of the new or unused pen needles are used and five used pen needle 5 get placed in the device 10 as shown in FIG. 12. Indeed, by exposing the spring 16 when all of the new or unused pen needles 5 are used, the user is provided with a visual indication that the device 10 is used-up. As this leaves the device 10 with no new or unused pen needles 5 and five used pen needles 5, the device 10 can be discarded with the therein-stored pen needle 5 safely arranged within the body 11.

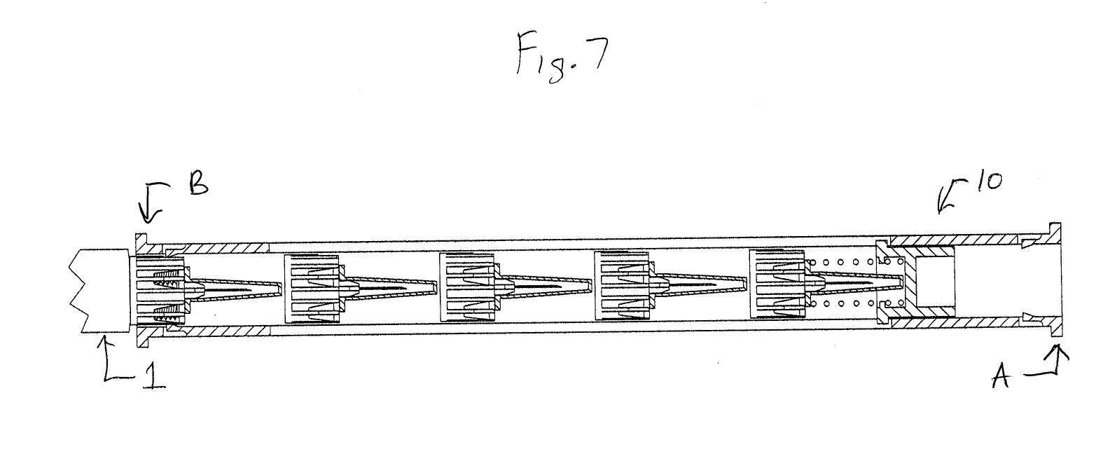

The device 10 of FIG. 7 is characteristic of another type of use configuration, i.e., having a state in which it is being used to install a first new pen needle 5 without the user first using the device 10 to remove a under pen needle 5. In this position, end A remains free of any used pen needles 5 while the end B receives therein a threaded section 2 of a device 1. When the user places the section 2 against the first new pen needle 5 and rotates either the device 1 or the device 11 along a direction that causes a threading-on of the pen needle 5 (until it reaches the installed position of FIG. 7), the user can then remove the device 1 from the device 10 and use the device 1 for injection. Once the first new pen needle 5 is removed from the device 10, the spring 16 will cause the second new pen needle 5 to move to a position in contact with the projections 12 as shown in FIG. 8.

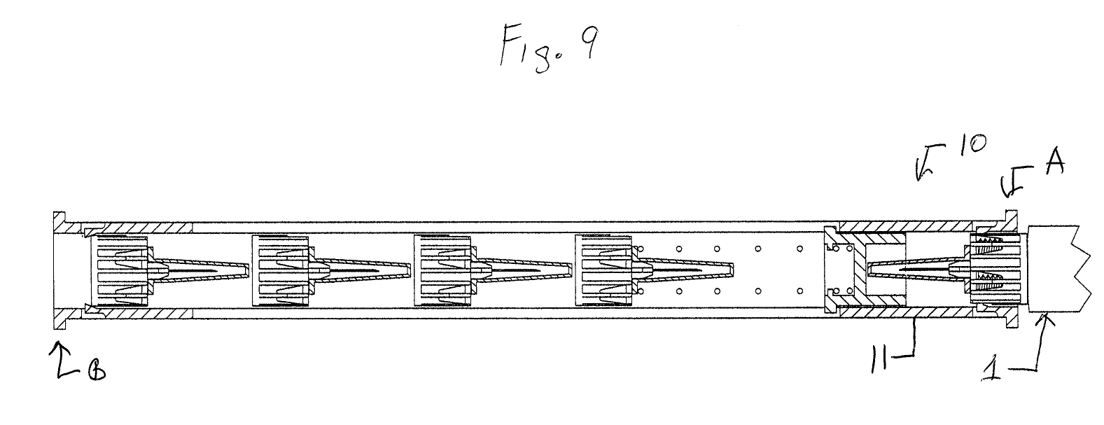

FIG. 9 shows a position after that of FIG. 8 wherein the end A receives therein the used and installed pen needle 5 previously removed from end B. When the user places the installed pen needle 5 (i.e., installed on the device 1) and then rotates either the device 1 or the device 10 along a direction that causes an unthreading of the pen needle 5 from the device 1, the device 10 can be used to safely remove the pen needle 5 from the device 1 as shown in FIG. 9. The removed pen needle 5 is then remains safely stored in end A of the body 11.

The device 10 of FIG. 10 shows a state in which it is being used to install a second new pen needle 5. In this position, the end B receives therein a threaded section 2 of a device 1. When the user places the section 2 against the first new pen needle 5 and rotates either the device 1 or the device 11 along a direction that causes a threading-on of the pen needle 5 (until it reaches the installed position of FIG. 11), the user can then remove the device 1 from the device 10 and use the device 1 for injection. Once the second new pen needle 5 is removed from the device 10, the spring 16 will cause the third new pen needle 5 to move to a position in contact with the projections 12.

The user can then use end A of the device 10 to remove the used pen needle 5 as discussed above. These steps are repeated until the device 10 for the last time is used to remove a used pen needle 5 as shown in FIG. 12. Once the last used pen needle 5 is safely disposed in end A, the device 10 can be safely disposed.

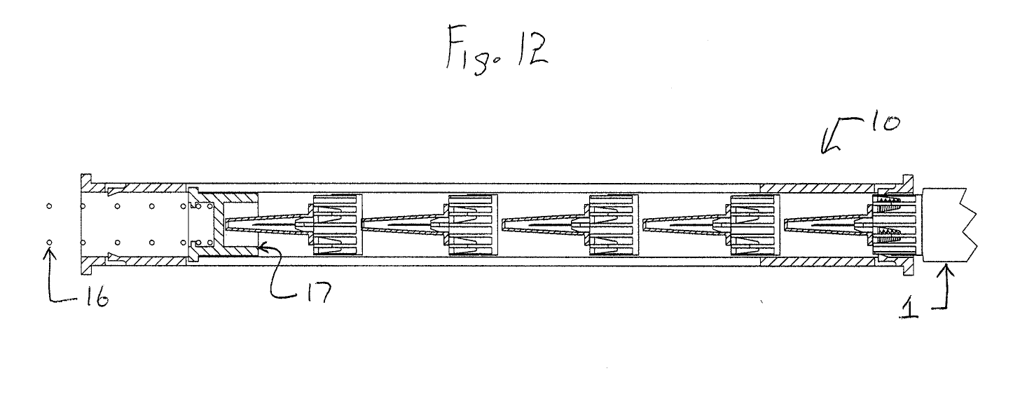

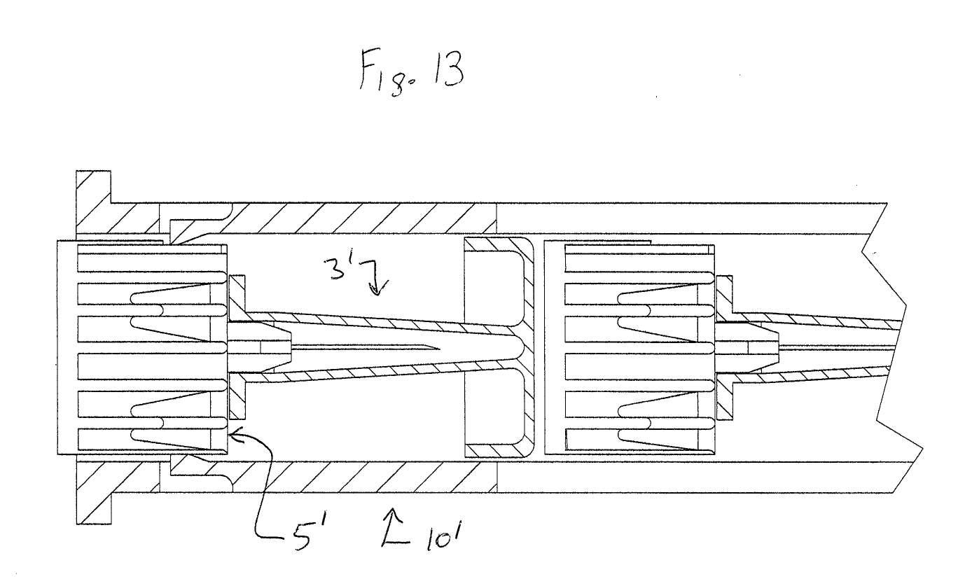

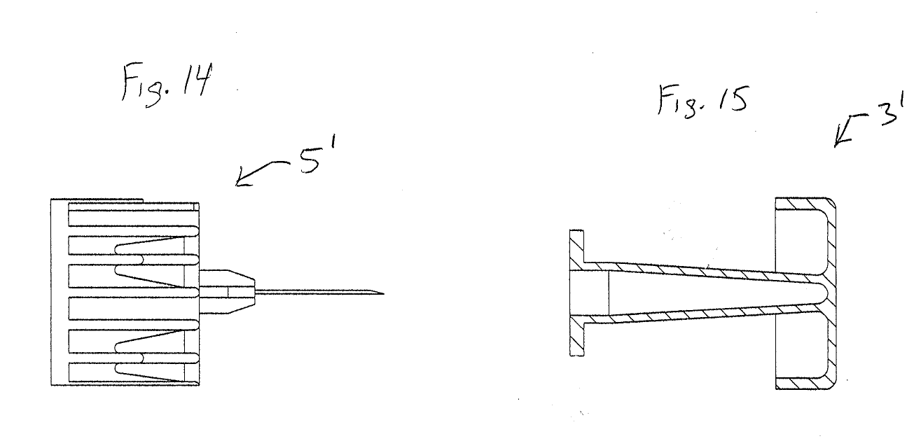

FIG. 13 shows an enlarged partial side cross-section view of a second non-limiting embodiment of a pen needle storage, installation and removal device according to the invention. The device 10′ is similar to that of the previous embodiment except that the pen needles 5′ utilize a differently configured removable needle cap 3′. As can be seen in FIGS. 14-17, each safety cap 3′ has a first end 3′a which is sized to slide over and frictionally engage with a needle hub of the pen needle 5′, a second end 3′b which is sized to slide within the space 14 of the body 11 and to be contacted and pushed distally by the body of an adjacent pen needle 5′, and a main body portion 3′c which is sized to slide over the needle of the pen needle 5′.

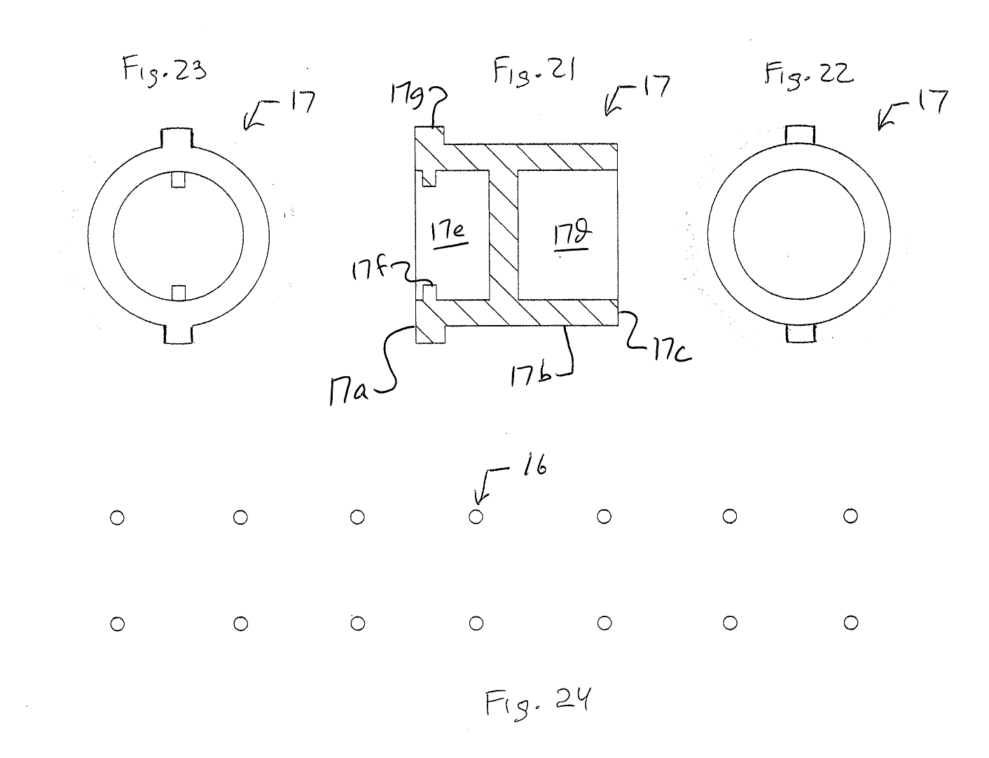

FIGS. 21-23 show side cross-section front end and rear end views of a pusher member 17 used in the first and second non-limiting embodiments of a pen needle storage, installation and removal device 10, 10′ according to the invention. The member 17 has a distal end 17a, a generally cylindrical main section 17b, and a proximal end 17c. The member 17 also has a proximal space which can accommodate a free end of the safety cover of a used pen needle 5, a distal open space 17e sized and configured to receive therein a proximal end of the spring 16, and one or more projections 17f sized and configured to axially retain or secure the proximal end of the spring 16. The member 17 also has one or more projections 17g sized and configured to extend into the slots 15 of the body 11 so as to guide the movement of the member 17 within the space 14 of the body 11.

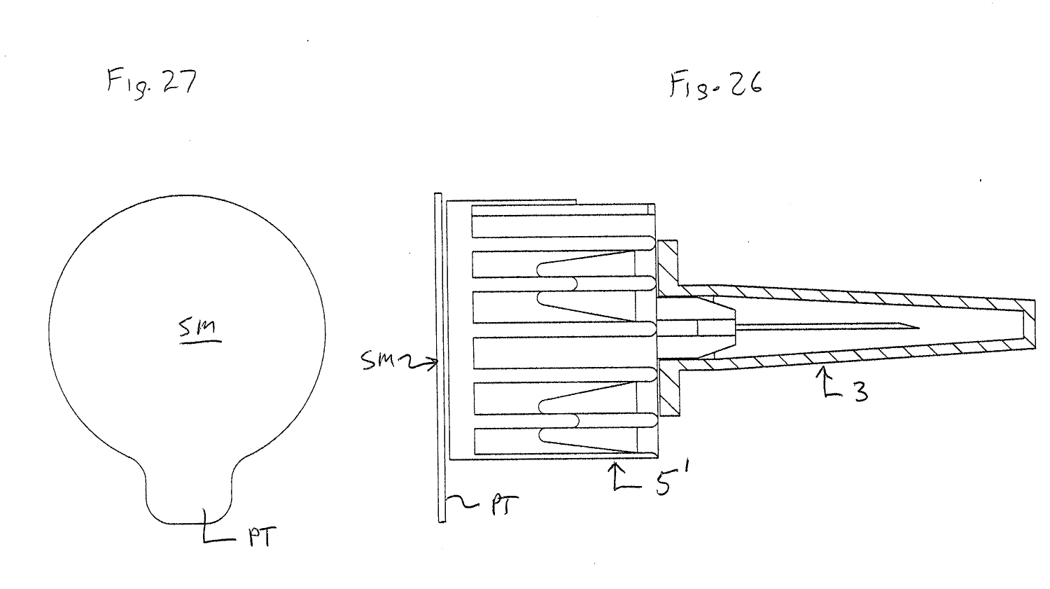

FIGS. 25-27 show side and end views of an embodiment of the pen needles 5′ that can be used in any of the herein disclosed devices. The pen needle 5′ is similar to those used in previous embodiments but additionally includes a removable sealing member SM having a pull tab PT. Although the pull tab PT is shown bent towards the body, it is preferably bent 180 degrees and away from the body of the pen needle 5′ so that the user can grip the same. The sealing member SM covers the open end of the body of the pen needle 5′ and is configured to prevent contamination. An adhesive formed on the sealing member SM ensures that it remains attached to a circumferential edge of the body of the pen needle 5′ until the user peels off the sealing member SM by pulling on the pull tab PT. When installed in the body 11, the free end of the cap 3 would contact the sealing member SM of an adjacent pen needle 5′ under the biasing force of the spring 16. When the pen needle 5′ is in a pre-installation position in the device 10, the user will first grip the pull tab PT inside end B and then remove the sealing member SM. Then, the user can insert the device 1 into end B as shown in FIG. 4. In this regard, the pull tab PT is preferably made long enough so that the user can grip the same even when the pen needle 5′ is in an installation position (in end B) similar to that shown in FIG. 8.

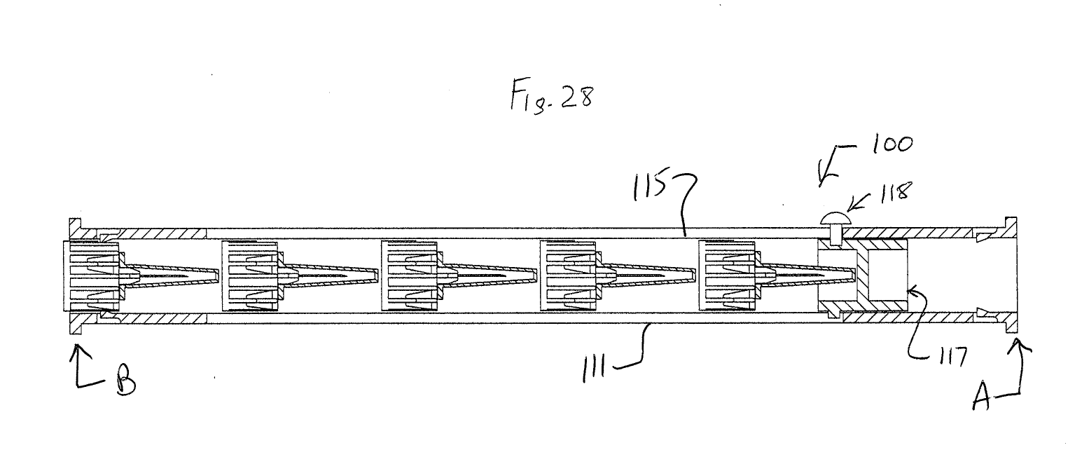

FIG. 28 shows a side cross-section view of another non-limiting embodiment of a pen needle storage, installation and removal device according to the invention. The device 100 is similar to that of FIG. 1 except that it utilizes a push-button 118 which allows a user to slide the member 117 towards end B in order to place the next new or unused pen needle 5 into a pre-installation position. In this embodiment, the spring 16 used in previous embodiments can be dispensed with. Of course, if desired, it is also possible to use such a spring on the device 100. The push-button 118 has an enlarged head which is arranged outside the body 111 and can be contacted and pushed by a user's thumb. The push-button 118 also has an end that extends into and is secured to the member 117 as well as a section arranged between the head and the member 117 that slides within the slot 115 of the body 111. The push-button 118 also provides a visual indication (based on its current position along the slot 115) to the user as to how many new or unused pen needles 5 remain in the device 100.

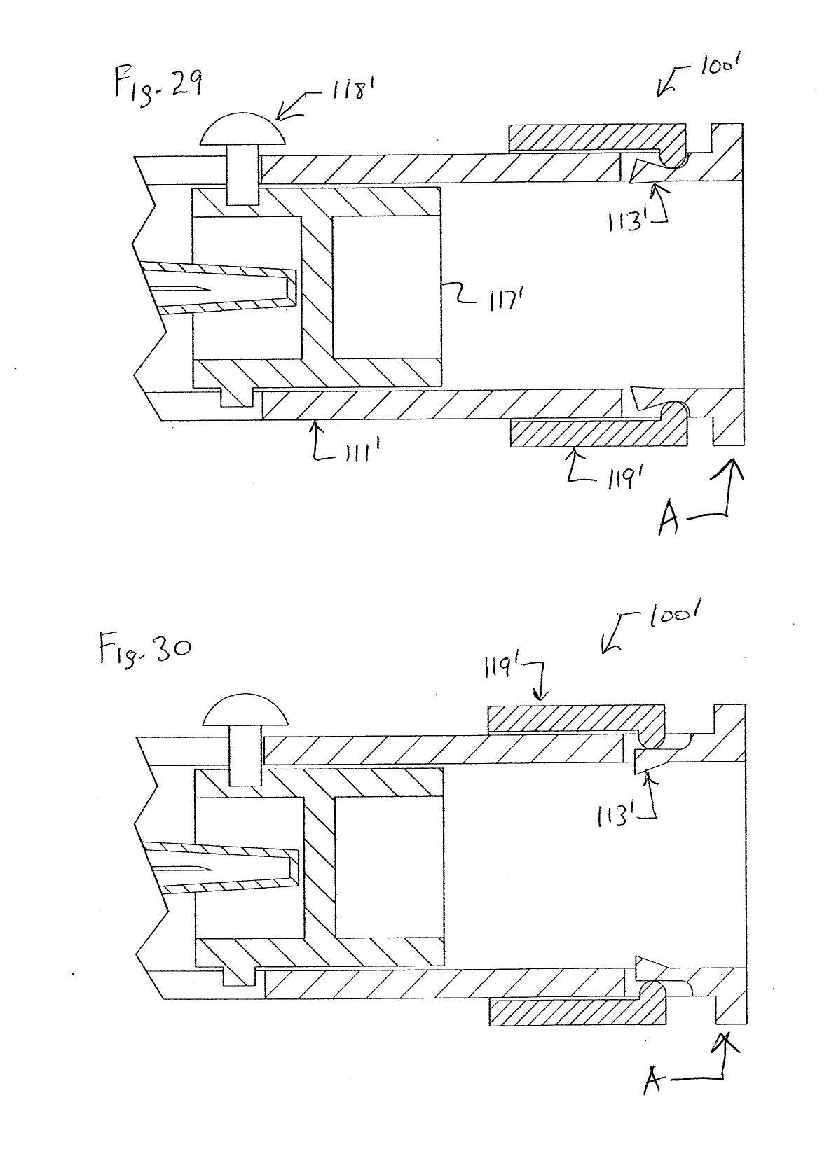

FIGS. 29 and 30 show partial side cross-section views of a removal end, i.e., end A, of another non-limiting embodiment of a pen needle storage, installation and removal device according to the invention. The device 100′ is similar to that of FIG. 28 except that it additionally utilizes a hand-actuated slide mechanism or sleeve 119′ which allows a user to move the engaging projections 113′ between a retracted or non-engaging position (FIG. 29) and an extended or engaging position (FIG. 30). FIG. 29 shows a pre-activation position of the sleeve 119′. When the user wishes to use the device 100′ to remove an installed used pen needle 5, the user positions it into end A (in the same way as shown in FIG. 2) and then moves the sleeve 119′ to the position shown in FIG. 30. As is apparent in FIGS. 29 and 30, the sleeve 119′ has two projections which each engage with one of the members 113′ such that in the position shown in FIG. 30 the projections 113′ are caused to deflect inwardly. However, when the user moves and/or allows the sleeve 119′ to move back to the position shown in FIG. 29, the members 113′ automatically move back to the retracted position.

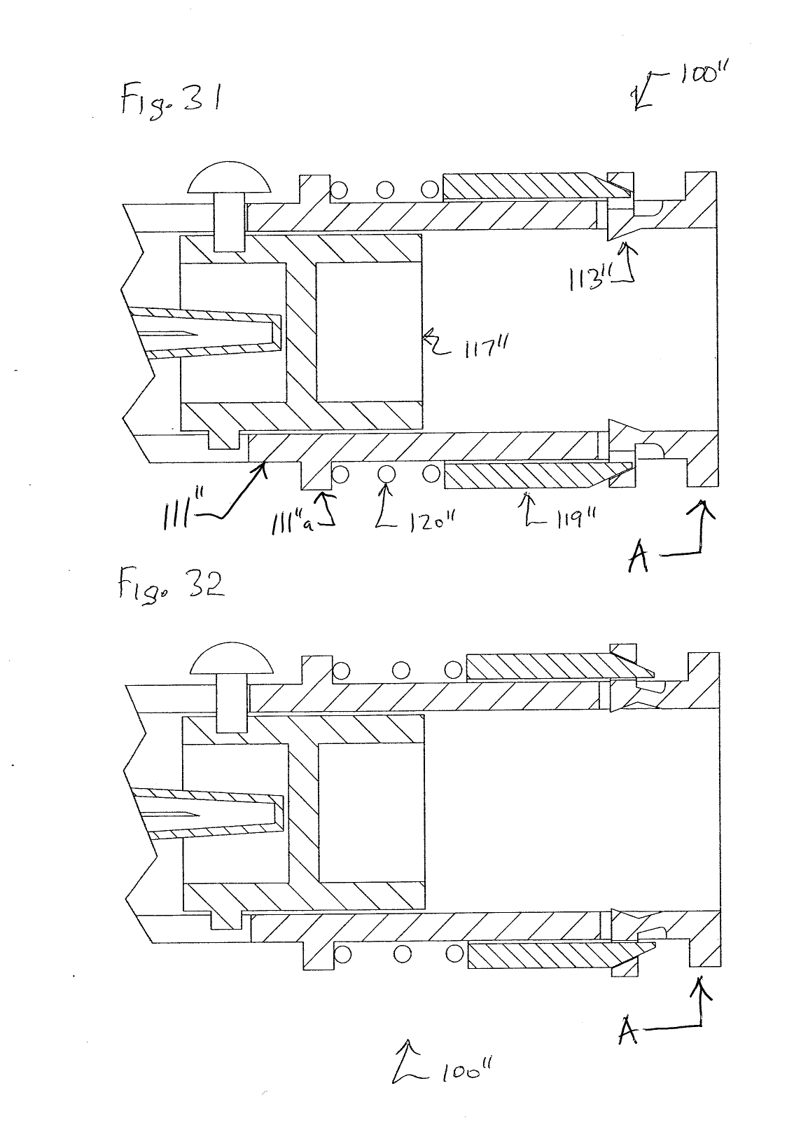

FIGS. 31 and 32 show partial side cross-section views of a removal end, i.e., end A, of another non-limiting embodiment of a pen needle storage, installation and removal device according to the invention. The device 100″ is similar to that of FIG. 28 except that it additionally utilizes a hand-actuated slide mechanism or sleeve 119″ which allows a user to move the engaging projections 113″ between a retracted or non-engaging position (FIG. 32) and an extended or engaging position (FIG. 31). FIG. 32 shows a pre-activation position of the sleeve 119″. When the user wishes to use the device 100′ to remove an installed used pen needle 5, the user positions it into end A (in the same way as shown in FIG. 2) and then moves the sleeve 119″ distally to the position shown in FIG. 31. As is apparent in FIGS. 31 and 32, the sleeve 119″ has two tapered proximal projections which each engage with and extend into a tapered opening formed in one of the members 113″ such that in the position shown in FIG. 32 the projections 113″ are caused to be deflected outwardly. However, when the user moves the sleeve 119″ to the position shown in FIG. 31 and against the biasing force of the spring 120″, the members 113″ automatically move back to the original or extended and engaging position. The spring 120″ is axially retained in a partially compressed state between a shoulder 111″a of the body 111″ and a distal end of the sleeve 119″.

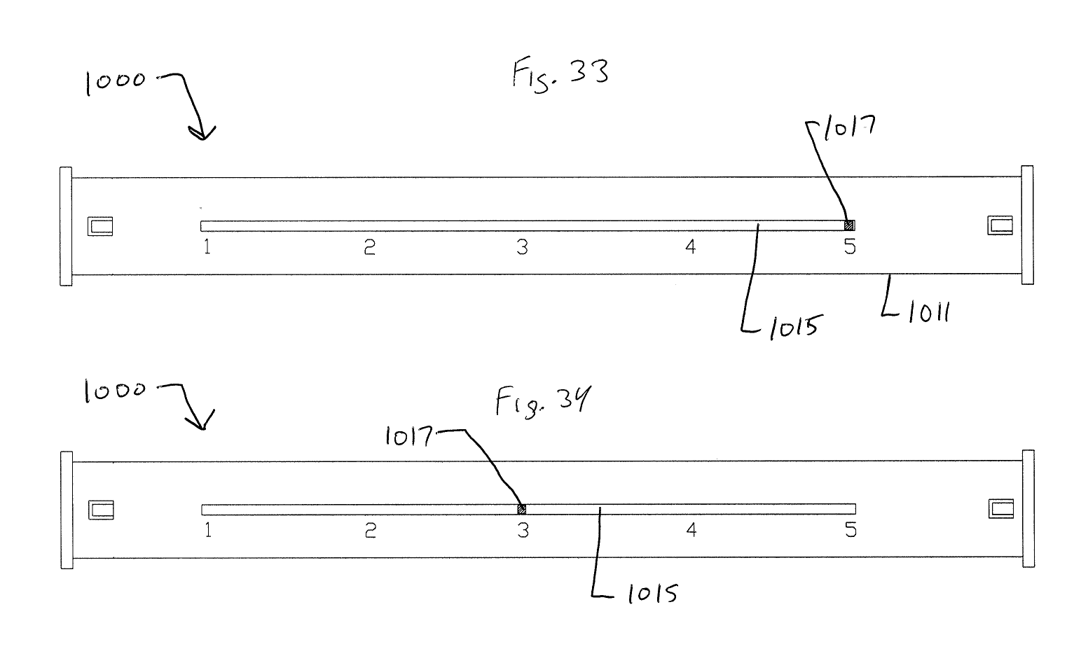

FIGS. 33 and 34 show another alternative embodiment of the device 1000. The device 1000 is similar to that of FIG. 1 except that the body 1011 includes indicia, e.g., numbers 1-5, which provide a visual indication to the user of how many new or unused pen needle 5 remain within the device 1000. Preferably, the pusher member 1017 (or at least the projection of the member 1017 which extends into the slot 1015) has a color that is different from that of the body 1011 so that the user can easily see the axial location of the member 1017 relate to the slot 1015. In FIG. 33, the member 1017 is located in a position indicating that five new or unused pen needles 5 remain in the device 1000. In FIG. 34, the member 1017 is located in a position indicating that three new or unused pen needles 5 remain in the device 1000. This configuration can be used on any of the herein disclosed embodiments. Alternatively or additionally, the body of anyone or more of the herein disclosed devices is made transparent or translucent so that the user can easily see how many new or unused pen needles 5 remain therein.

The devices described herein can also utilize one or more features disclosed in prior art documents expressly incorporated by reference in pending U.S. patent application Ser. No. 11/616,195 (Publication No. 2008/0154192). This application and the documents expressly incorporated therein is hereby expressly incorporated by reference in the instant application. Furthermore, one or more of the various parts of the device can preferably be made as one-piece structures by e.g., injection molding, when doing so reduces costs of manufacture. Non-limiting materials for most of the parts include synthetic resins such as those approved for syringes, blood collection devices, or other medical devices. Furthermore, the invention also contemplates that any or all disclosed features of one embodiment may be used on other disclosed embodiments, to the extent such modifications function for their intended purpose. purpose.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A device for at least one of storing, installing and removing pen needles, the device comprising:
a body configured to store at least one pen needle;
a first end of the body being configured to install a pen needle on an injection device; and
a second end of the body being configured to remove a pen needle from the injection device.

2. The device of claim 1, wherein the at least one pen needle comprises at least two pen needles.

3. The device of claim 1, wherein the at least one pen needle comprises at least three pen needles.

4. The device of claim 1, wherein the at least one pen needle comprises between four and eight pen needles.

5. The device of claim I, wherein the body is generally cylindrical.

6. The device of claim 1, wherein the first end of the body comprises at least one mechanism for releasably retaining the at least one pen needle which releasably retains the at least one pen needle until it is fully installed on the injection device.

7. The device of claim 1, wherein the first end of the body comprises plural mechanisms for releasably retaining the at least one pen needle which releasably retains the at least one pen needle until it is fully installed on the injection device.

8. The device of claim 1, wherein the first end of the body comprises at least one mechanism for releasably retaining the at least one pen needle in an installation position.

9. The device of claim 1, wherein the first end of the body comprises plural mechanisms for releasably retaining the at least one pen needle in an installation position.

10. The device of claim 1, wherein the second end of the body comprises at least one mechanism for releasably retaining the at least one pen needle during removal of the pen needle from the injection device.

11. The device of claim 1, wherein the first end of the body comprises plural mechanisms for releasably retaining the at least one pen needle during removal of the pen needle from the injection device.

12. The device of claim 1, wherein the second end of the body comprises at least one mechanism for releasably retaining the at least one pen needle in a removal position.

13. The device of claim 1, wherein the first end of the body comprises plural mechanisms for releasably retaining the at least one pen needle in a removal position.

14. The device of claim 1, wherein the first end of the body is configured to retain the at least one pen needle while a needle of the at least one pen needle is oriented into the body.

15. The device of claim 1, wherein the first end of the body is configured to retain the at least one pen needle while a needle of the at least one pen needle is oriented towards the second end.

16. The device of claim 1, wherein the second end of the body is configured to retain the at least one pen needle while a needle of the at least one pen needle is oriented out of the body.

17. The device of claim 1, wherein the second end of the body is configured to retain the at least one pen needle while a needle of the at least one pen needle is oriented opposite the first end.

18. The device of claim 1, wherein the first end of the body is configured to retain a new or unused pen needle.

19. The device of claim 1, wherein the second end of the body is configured to retain a used pen needle.

20. The device of claim 1, wherein the body is configured to retain a new or unused pen needle in the first end while the at least one pen needle is arranged between the first and second ends.

21. The device of claim 1, wherein the body is configured to retain a used pen needle in the second end while the at least one pen needle is arranged between the first and second ends.

22. The device of claim 1, wherein, while the at least one pen needle is arranged between the first and second ends, the body is configured to:
retain a new or unused pen needle in the first end; and
retain a used pen needle in the second end.

23. The device of claim 1, wherein, while the at least one pen needle is arranged between the first and second ends, the body is configured to:
releasably retain a new or unused pen needle in the first end; and
non-removably retain a used pen needle in the second end.

24. A method of installing a pen needle on an injection device using the device of claim 1, the method comprising:
arranging the first end adjacent a pen needle receiving end of the injection device; and
moving the device towards the injection device until the pen needle is installed thereon.

25. A method of removing a pen needle from an injection device using the device of claim 1, the method comprising:
arranging the second end adjacent a pen needle receiving end of the injection device; and
moving the device towards the injection device until the pen needle is installed in the second end.

26. A method of installing a pen needle on an injection device using the device of claim 1, the method comprising:
arranging the first end adjacent a pen needle receiving end of the injection device;
moving the device towards the injection device until the pen needle is installed thereon; and
moving the device away from the injection device with one fewer pen needle being arranged in the body than prior to the arranging.

27. A method of removing a pen needle from an injection device using the device of claim 1, the method comprising:
arranging the second end adjacent a pen needle receiving end of the injection device;
moving the device towards the injection device until the pen needle is installed in the second end; and
moving the device away from the injection device with one additional pen needle being arranged in the body than prior to the arranging.

28. A device for storing and at least one of installing and removing pen needles, the device comprising:
an elongate body configured to store plural pen needles;
a first end of the body being configured to install a pen needle on an injection device; and
a second end of the body being configured to remove a pen needle from the injection device.

29. A device for storing, installing and removing pen needles, the device comprising:
a body configured to store plural pen needles;
a first end of the body being configured to install a pen needle on an injection device;
a second end of the body being configured to remove a pen needle from the injection device; and
a movable device arranged in the body adapted to separate at least one unused pen needle from a used pen needle.

30. A device for at least one of storing, installing and removing skin puncturing devices, the device comprising:
a body configured to store at least one skin puncturing device;
a first end of the body being configured to install a skin puncturing device; and
a second end of the body being configured to remove a skin puncturing device.

31. The device of claim 30, wherein the at least one skin puncturing device comprises at least one pen needle.

32. The device of claim 30, wherein the device is disposable after being used at least one time.

33. The device of claim 30, wherein the device is rendered un-usable after being used a predetermined number of times.

34. The device of claim 30, wherein the device is re-fillable after being used at least one time.
